(19) United States
(12) Patent Application Publication
(10) Pub. No.: US 2024/0382213 A1
(43) Pub. Date: Nov. 21, 2024

(54) MEDICAL CUTTING INSTRUMENT INCLUDING BURR GUIDE PROTECTOR

(71) Applicant: TECHCORD CO., LTD., Daejeon (KR)

(72) Inventor: Youngkyu CHOI, Daejeon (KR)

(21) Appl. No.: 18/624,742

(22) Filed: Apr. 2, 2024

(30) Foreign Application Priority Data
May 17, 2023 (KR) 10-2023-0063892

(51) Int. Cl. A61B 17/16; A61B 17/15; A61B 17/17

(52) U.S. Cl. CPC A61B 17/1613; A61B 17/15; A61B 17/17

(57) ABSTRACT

The present invention provides a medical burr comprising: a rotating unit that is formed to have a smaller diameter than a guide that extends long forward in a medical cutting instrument, and rotates while extending inside the guide; and a cutting unit formed at the tip of the rotating unit, and further comprising a guide protector that surrounds the exposed portion of the burr.

### TECHNICAL FIELD

The present invention relates to a medical cutting instrument that includes a burr guide protector and thus ensures a wide field of view for the surgical site.

### BACKGROUND ART

Cutting instruments such as burrs and saws are connected to handpieces of the electric equipment, and used to cut and polish bones and tissues during examination and surgeries. Such cutting instruments have recently been attracting attention as one of the spinal surgery methods used in bidirectional spinal endoscopic surgery. FIG. 1 shows some examples of burrs that are currently commercially available.

Korean Patent No. 10-2348589 discloses a burr connection structure for spinal surgery in which the bur connection structure assembles and connects the rear end of a surgical bur 10 of a predetermined length having a cutting blade 11 disposed at the front end part to a drill hole provided in a driving unit body 60′, and the surgical bur 10 is inserted to be rotatable and supported inside a guide rod 20, as illustrated in FIG. 2. The drive unit body 60′ is configured such that an adapter 50′ is coupled thereto in a fitting form.

Burrs that are currently in use include an open type as shown in FIG. 3A and a semi-open type as shown in FIG. 3B. The open type is useful for use in locations where the cutting site is open, but is inappropriate for use in locations where the cutting site is in the inside and surrounded by tissue, nerves or the like. FIG. 3C conceptually illustrates an example in which tissue or nerves may also be cut instead of the object to be cut when an open-type burr is used.

The semi-open type has a structure suitable for use even where the cutting site is opened or even when the cutting site is in the inside. Since most spine surgery is minimally invasive surgeries rather than surgeries that are performed by completely opening a surgical site, the cutting site is mostly located in the inside and thus, the type illustrated in FIG. 3B is often used. This is a type of tube in which one side of the tube is opened so that only the desired site can be cut during cutting, and is equipped with a large-diameter pipe-like protector 30′ that extends long enough to cover up to a portion of a guide rod 20′, a burr 10′, and a cutting blade 11′. The protector 30′ protects the upper nerves and tissues that are not to be cut. However, since the field of view of the surgeon performing the surgery is covered by the large protector 30′ so large that the entire cutting site cannot be seen, there is a problem that it is not possible to accurately confirm whether the cutting is done in the desired site.

### DETAILED DESCRIPTION OF THE INVENTION

### Technical Problem

It is an object of the present invention to provide a medical cutting instrument that includes a burr guide protector and thus ensures a wide field of view for the surgical site.

It is another object of the present invention to provide a medical cutting instrument having improved safety in use because a tube is formed like spherical burrs in medical cutting instrument burr products.

### Technical Solution

In order to achieve the above-mentioned object, according to the present invention, there is provided a medical burr comprising: a rotating unit that is formed to have a smaller diameter than a guide that extends long forward in a medical cutting instrument, and rotates while extending inside the guide; and a cutting unit formed at the tip of the rotating unit, and further comprising a guide protector that surrounds the exposed portion of the burr, wherein the guide protector includes a base that extends forward from the tip of the guide and surrounds the rotating unit, and a protection unit formed at the tip of the base and surrounding the cutting unit, thereby making it possible to ensure the practitioner's field of view and protect the non-cutting sites of the body through the guide protector.

The base is a flat plate or a concave-shaped plate that extends adjacent to the rotating unit, and can be made into a size of width that covers one surface of the rotating unit so as to protect the body site rather than an object being cut.

The protection unit may be made to ensure the operator's field of vision so that the cutting blade of the cutting unit does not touch the non-cutting site of the body.

The cutting unit has a spherical shape, and the protection unit may have a curved surface shape and rise gently toward the cutting site.

The cutting unit may have a tip shape, and the protection unit may have a flat plate shape.

The cutting unit may have a drill shape, and the protection unit may have a flat plate shape.

Part of the guide protector may be made of a material that is not damaged or worn even when coming into contact with the rotating unit or the cutting unit.

### Advantageous Effects

The present invention changes the shape of conventional surgical instruments, and thereby, can prevent problems that may arise during surgery, e.g., surgical after-effects and medical accidents caused by cutting non-problematic sites such as nerves and tissues, and can exhibit the effects of reducing fatigue and increasing the efficiency of surgery.

### BRIEF DESCRIPTION OF THE DRAWINGS

FIG. 1 shows some examples of burrs that are currently commercially available;

FIG. 2 is a diagram illustrating a burr connection structure for spine surgery of a prior patent;

FIGS. 3A-3C are diagrams illustrating a burr that is currently in use;

FIG. 4 is an overall side view of a medical cutting instrument including a burr of the present invention;

FIG. 5A is a side view of a burr equipped with a guide protector;

FIG. 5B is a plane view; and

FIG. 5C is a perspective view;

FIG. 6A is a side view of a burr according to another embodiment of the present invention; and

FIG. 6B is a top view of a burr according to another embodiment of the present invention;

FIG. 7A is a side view of a burr according to another embodiment of the present invention.

FIG. 7B is a top view of a burr according to another embodiment of the present invention.

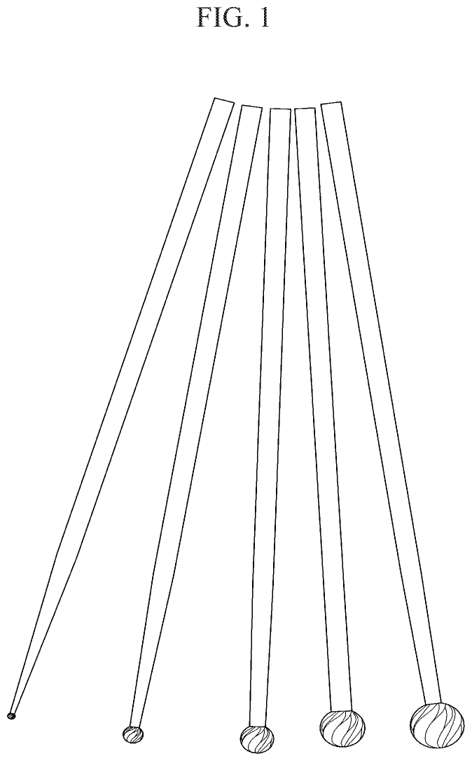

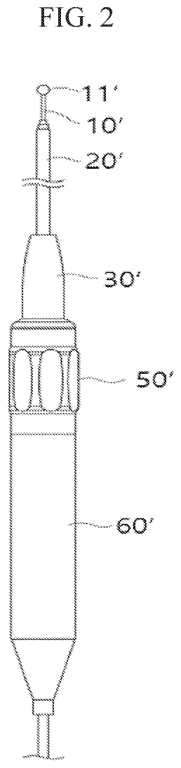

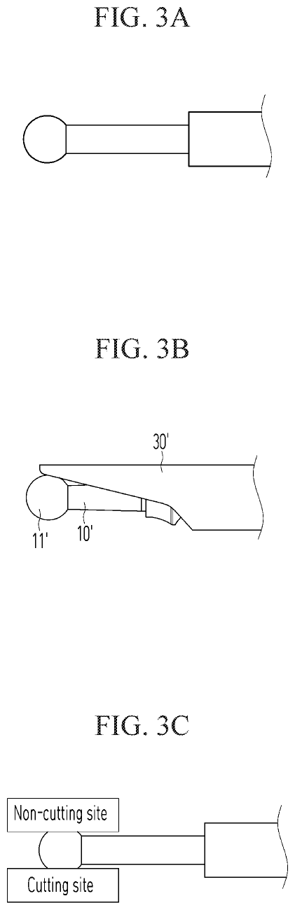

### DETAILED DESCRIPTION OF THE EMBODIMENTS

Each embodiment according to the present invention is merely an example to aid understanding of the present invention, and the present invention is not limited to such embodiments. The present invention may be configured by a combination of at least one or more of the individual components and individual functions included in each embodiment.

The present invention provides a structure that not only takes the form of a medical cutting instrument burr and has the form of a tube which is a guide protector to secure the field of view, but also prevents nerves, tissues, and the like from being cut in the opposite direction while cutting in a desired direction.

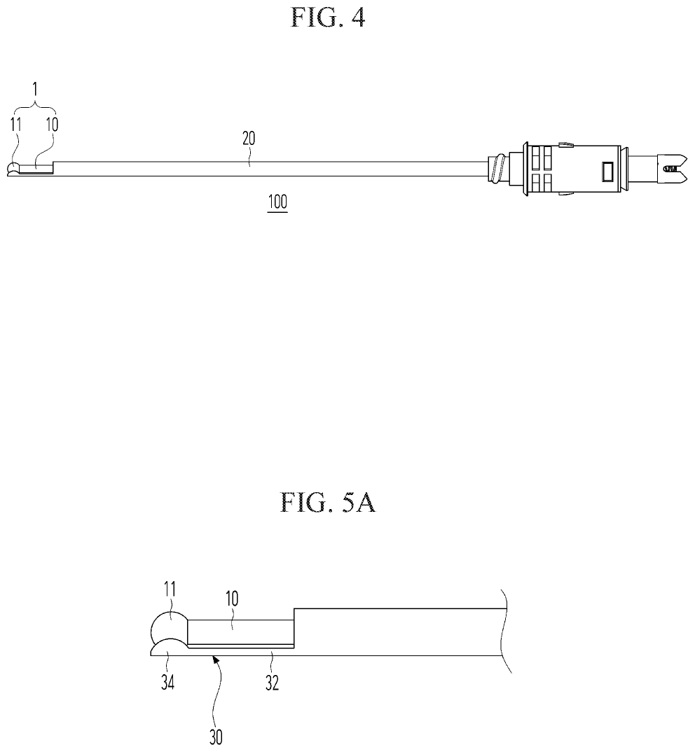

FIG. 4 is an overall side view of a medical cutting instrument 100 including a burr 1 of the present invention. A guide 20 extends long from a driving unit (not shown) of the medical cutting instrument 100, and the burr 1 of the present invention is mounted on the guide 20. The scope of the burr 1 can be defined in several ways, but the burr 1 of the present invention basically includes a rod-shaped rotating unit 10 that is formed to have a smaller diameter than a guide 20, and extends inside the guide 20 and is connected to the driving unit to rotate, and a cutting unit 11 formed at the tip of the rotating unit.

The configuration of the medical cutting instrument 100 described above is merely an example, and may also include the burr 1 with any structure of the prior art.

The main feature of the present invention resides in the installation of a guide protector 30 that surrounds the exposed portion of the burr 1.

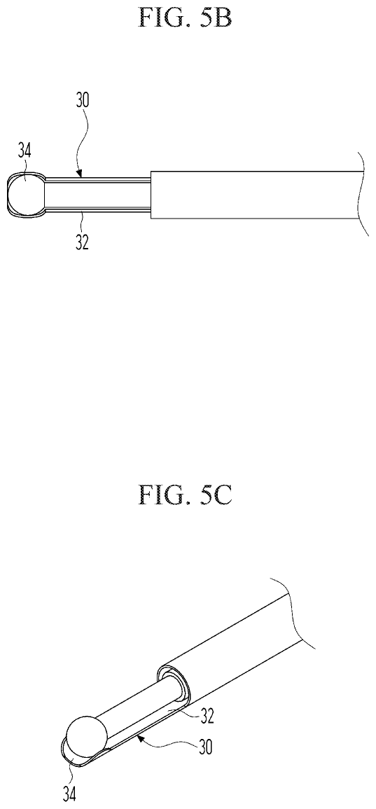

FIG. 5A is a side view of a burr 1 equipped with a guide protector 30, FIG. 5B is a plane view, and FIG. 5C is a perspective view. The explanation will be given on the assumption that nerves or tissues, which are non-cutting sites, are located on the lower surface of the burr 1.

The guide protector 30 includes a base 32 that extends forward from the tip of the guide 20 and surrounds the rotating unit 10, and a protection unit 34 formed at the tip of the base 32 and surrounding the cutting unit 11. In the present invention, the meaning of “surrounding” or “wrapping” or “covering” should be interpreted in a broad sense, including not only being adjacent to another member but also contacting a part of another member.

The base 32 is a flat plate or a slightly concave shaped plate, extends adjacent to the rotating unit 10, and is made to have a width that completely covers one side surface, that is, the lower surface, of the rotating unit 10 when viewed from a plan view. When the inner surface of the base 32 is made to come into contact with the rotating unit 10, it must be made of a material strong enough to not cause wear or damage. In order to ensure the operator's field of view to the maximum, it is preferable that the height of the base 32 is not large.

The protection unit 34 must be made in a structure that ensures the operator's field of view to the maximum while preventing the cutting blade of the cutting unit 11 from touching non-cutting sites of the body. In the case of a spherical cutting unit 11 as illustrated, since the whole of the outer sites of the sphere becomes a cutting surface, the side surface is curved and somewhat gently raised so that the protection unit 34 sufficiently protects the non-cutting site. However, in this case as well, it is preferable that the height is less than the radius of the cutting unit 11 in order to secure the field of view. Wrapping the spherical cutting unit 11 upward to some extent as illustrated in FIGS. 5A-5C is useful when the non-cutting site is located not only at the lower part but also at the side surface. The width of the protection unit 34 is made into a size that completely covers the lower surface of the cutting unit 11 in a plan view.

When the inner surface of the protection unit 32 comes into contact with the cutting unit 11, it must be made of a material strong enough to not cause wear or damage. When the cutting unit 11 abuts on the body tissue and is pressed during actual procedure, the inner surface of the protection part 32 comes into contact with the cutting unit 11, and thus, it is necessary to ensure sufficient strength.

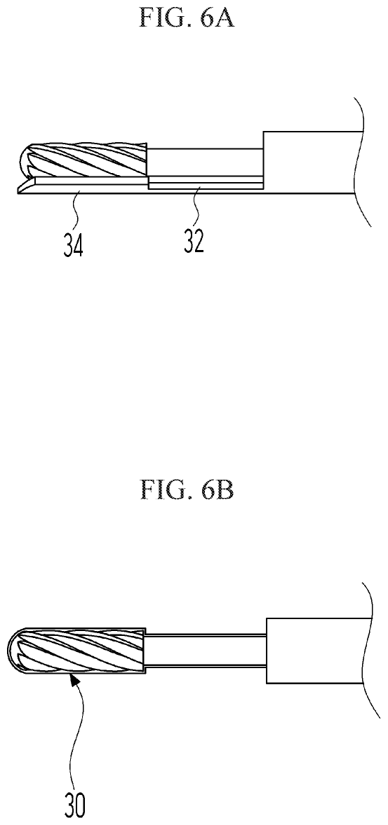

FIG. 6A is a side view of a burr 1 according to another embodiment of the present invention.

FIG. 6B is a top view of a burr 1 according to another embodiment of the present invention.

The cutting unit 11 of the burr 1 has a drill shape. The base 32 and the protection unit 34 of the guide protector 30 are made into a flat shape, and are made so as to completely cover the lower part and to have a width that covers the lower surfaces of the rotating unit 10 and the cutting unit 11.

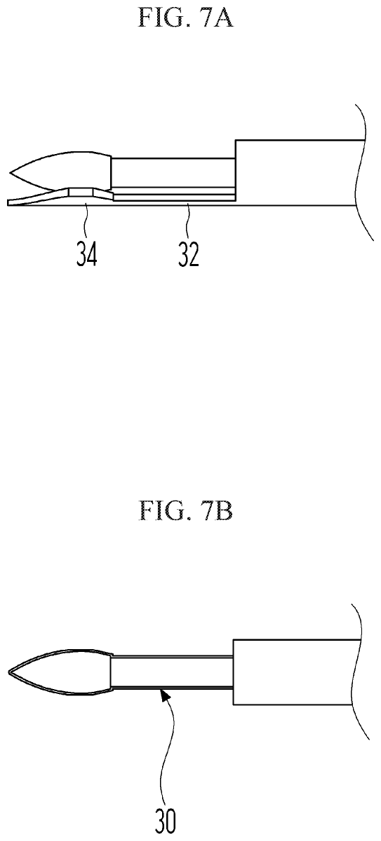

FIG. 7A is a side view of a burr 1 according to another embodiment of the present invention.

FIG. 7B is a top view of a burr 1 according to another embodiment of the present invention.

The cutting unit 11 of the burr 1 has a tip shape with a sharp tip. The base 32 and the protection unit 34 of the guide protector 30 are made into a flat shape, and are made so as to completely cover the lower part and to have a width that cover the lower surfaces of the rotating unit 10 and the cutting unit 11.

In the present invention, the protection unit 34 completely covers and protects the lower surface of the cutting unit 11, but a size exceeding the cutting unit 11 is not preferable, and it is preferable to form it so as to follow a spherical shape, a drill shape, and a tip shape as illustrated in the drawings.

The present invention is a surgical instrument that is often used in neurosurgery, orthopedics, and the like, but can be applied to many fields.

The present invention changes the shape of conventional surgical instruments, and thereby, can prevent problems that may arise during surgery (e.g., surgical after-effects and medical accidents caused by cutting non-problematic sites such as nerves and tissues), and can exhibit the advantages of reducing fatigue and increasing the efficiency of surgery.

While the present invention has been described in detail with reference to specific embodiments and illustrative drawings, the present invention is not limited thereto, and it will be apparent to those skilled in the art that various changes and modifications can be made without deviating from the spirit and scope of the present invention.

1. A medical burr comprising: a rotating unit that is formed to have a smaller diameter than a guide that extends long forward in a medical cutting instrument, and rotates while extending inside the guide; and a cutting unit formed at the tip of the rotating unit, and further comprising a guide protector that surrounds the exposed portion of the burr,
wherein the guide protector includes a base that extends forward from the tip of the guide and surrounds the rotating unit, and a protection unit formed at the tip of the base and surrounding the cutting unit, thereby making it possible to ensure the practitioner's field of view and protect the non-cutting sites of the body through the guide protector.

2. The medical burr as claimed in claim 1, wherein:
the base is a flat plate or a concave-shaped plate that extends adjacent to the rotating unit, and can be made into a size of width that covers one surface of the rotating unit so as to protect the body site rather than an object being cut.

3. The medical burr as claimed in claim 2, wherein:
the protection unit is made to ensure the operator's field of vision so that the cutting blade of the cutting unit does not touch the non-cutting site of the body.

4. The medical burr as claimed in claim 3, wherein:
the cutting unit has a spherical shape, and the protection unit has a curved surface shape and rises gently toward the cutting site.

5. The medical burr as claimed in claim 3, wherein:
the cutting unit has a tip shape, and the protection unit has a flat plate shape.

6. The medical burr as claimed in claim 3, wherein:
the cutting unit has a drill shape, and the protection unit has a flat plate shape.

7. The medical burr as claimed in claim 3, wherein:
part of the guide protector is made of a material that is not damaged or worn even when coming into contact with the rotating unit or the cutting unit.
